(19) United States
(12) Patent Application Publication
(10) Pub. No.: US 2015/0024448 A1
(43) Pub. Date: Jan. 22, 2015

(54) Integrated Processes for the Conversion of Coal to Chemicals

(71) Applicants: Mansour Djadali, Coasta Mesa, CA (US); Norman Louis Balmer, Ridgefield, CA (US)

(72) Inventors: Mansour Djadali, Coasta Mesa, CA (US); Norman Louis Balmer, Ridgefield, CA (US)

(21) Appl. No.: 14/331,338

(22) Filed: Jul. 15, 2014

Related U.S. Application Data
(60) Provisional application No. 61/957,947, filed on Jul. 16, 2013.

(51) Int. Cl. C12P 7/08; C12P 7/16

(52) U.S. Cl. CPC C12P 7/08; C12P 7/16
USPC 435/160; 435/163

(57) ABSTRACT

Integrated processes are disclosed for the conversion of coal containing mercury compounds, hydrocarbon-containing compounds, sulfur-containing compounds and nitrogen-containing compounds to chemicals in which coal is heated to remove mercury components, pyrolyzed to remove sulfur-containing and nitrogen-containing compounds and then gasified to provide syngas, and syngas is bioconverted to liquid product.

### RELATED APPLICATIONS

Priority is claimed to provisional patent application 61/957,947, filed Jul. 16, 2013, hereby incorporated by reference in its entirety.

### FIELD OF THE INVENTION

This invention pertains to integrated processes having a high, total carbon efficiency for converting coal to chemicals including, but not limited to, liquid fuels and oxygenated organic compounds. The processes thermally treat coal to remove certain components, and then, integrated with the thermal treatment, the coal is gasified to generate a synthesis gas (syngas) that is bioconverted to chemicals.

### BACKGROUND

Coal is an abundant source of energy. However, its solid form is not conducive to meet growing demands for fuels for powering transport vehicles and vessels (“fuel for mobile applications”) that are now being derived from crude oil. Additionally pollutants such as nitrogenous compounds, sulfur-containing compounds and heavy metals are present in coal. Thus, the primary uses of coal as a source of energy have been limited to large facilities for burning coal to produce heat or electricity where transporting and handling coal can be cost justified as well as the installation and operation of equipment to remove pollutants.

Coal has not been a significant resource to meet the demand for chemicals. Processes do exist for liquefaction of coal and for converting coal to liquids via the Fischer Tropsch process, but both processes suffer from limitations, particularly high capital and operating costs. Accordingly, processes are desired for the conversion of coal to chemicals in an efficient, economically attractive and environmentally acceptable manner.

Proposals have been made to gasify carbonaceous fuel to provide a syngas for anaerobic fermentation to produce biochemicals or biofuels such as ethanol and butanol. The bioconversion can be relatively selective to the sought bioproduct. Any integrated gasification and syngas bioconversion process has complex interactions, and with the use of coal, the interactions are even more pronounced. For instance, sulfur and nitrogen containing components in the coal and volatile metal components in the coal such as mercury can result in a syngas containing components that may be adverse to microorganisms. Removal of these components can add to the capital and operating costs of the process. Additionally, the concentration of carbon monoxide and hydrogen in the syngas can affect the rate of their mass transfer into the aqueous fermentation medium for conversion by the microorganisms. Any diluents present in feeds to, or produced by, the gasification process can thus affect the fermentation process. Thus, air blown, direct fired gasifiers pose challenges in that the syngas would be diluted with nitrogen. Oxygen blown, direct fired gasifiers, while essentially eliminating the nitrogen dilution of the syngas, require the use of cryogenic oxygen plants which are capital and energy intensive. Indirectly fired gasifiers require a source of fuel to combust to provide heat.

Accordingly, improved processes are sought for the conversion of coal to chemicals that minimize capital and operating costs, provide desirable syngas compositions and provide high total carbon conversions in an environmentally-acceptable manner.

### SUMMARY

By this invention integrated processes are provided for the pretreatment of coal, gasification of coal to produce syngas and the fermentation of syngas to chemicals, especially oxygenated organic compounds. This integration in accordance with the invention provides capital and operating economic efficiencies. In preferred aspects of the invention, the integrated processes enable the use of low rank coals, especially subbituminous coal, that are less expensive than bituminous and anthracite coal.

The integrated processes of this invention comprise a thermal treatment of the coal prior to being introduced into the gasifier wherein the thermal treatment materially reduces the concentration of sulfur and nitrogen compounds in the coal and substantially reduces the concentration of volatile metal compounds in the coal. The thermal treatment enables a syngas to be produced that does not require undue cleanup operations in order to be used as substrate for anaerobic fermentation to produce liquid products. Further, the thermal pretreatment provides a hydrocarbonaceous vapor phase that can be used to provide liquid fuel or feedstock and/or can be used as at least a portion of the fuel to a fired heater to provide indirect heat to the gasifier. Heat for the thermal treatment of the coal is provided at least in part by indirect heat exchange. Where the hydrocarbons vapor phase is combusted to provide heat, the sulfur and nitrogenous compounds are oxidized and can be more facilely recovered by conventional flue gas processes.

In some instances, the indirect heat provided to the gasifier can substantially reduce, and in some instances eliminate, the need for direct firing in the gasifier. Hence, the capital and operating expense for a cryogenic oxygen plant to provide oxygen for oxygen blowing of the gasifier can be reduced or avoided while providing desirable concentrations of carbon monoxide and hydrogen in the syngas product for the fermentation without undue dilution with nitrogen or undue amounts of hydrogen cyanide and other nitrogen-containing compounds being produced. Where the gasifier requires direct firing, air or, preferably, oxygen-enriched air may be used as the oxidant. Since a portion of the heat for the gasification is provided by indirect heating, the nitrogen dilution of the syngas can be maintained at acceptable levels.

In one broad aspect of this invention, processes are provided for the conversion of coal containing mercury compounds, hydrocarbon-containing compounds, sulfur-containing compounds and nitrogen-containing compounds to chemicals comprising:
a. drying particulate coal, preferably having a major dimension less than about 50 millimeters, preferably less than about 5 or 10 millimeters, at a temperature less than about 200° C. resulting in a water vapor overhead;
b. heating the dried coal to a temperature to between about 200° C. and 320° C. to remove mercury from the coal by volatilization and provide a heated coal;
c. thermally treating the heated coal at a temperature between about 430° C. to about 700° C. for a time sufficient to release vaporous organic compounds to provide a pyrolyzed coal and a hot vapor stream containing hydrocarbon-containing compounds, said thermal treatment being sufficient to reduce the concentration of sulfur-containing and nitrogen-containing components by release from the coal being thermally treated;
d. combusting at least a portion of the hot vapor stream of step (c) to provide a hot exhaust stream wherein prior to combusting, the hot vapor stream is maintained at a temperature sufficient to avoid condensate from forming, preferably said at least a portion of the hot vapor stream is maintained substantially at or above the temperature of the thermal treating until combusted;
e. gasifying the pyrolyzed coal in the presence of steam under gasification conditions, said gasification conditions preferably comprising a temperature in the range of between about 800° C. and 1200° C. and a steam to carbon mole ratio of about 1.1:1 to 4:1, to provide an ash fraction and a syngas, wherein at least a portion of heat energy in the hot exhaust stream of step (d) provides indirect heat for gasification;
f. cooling by indirect heat exchange the syngas from step (e) and generate steam;
g. passing at least a portion of the cooled syngas to at least one fermenter containing an aqueous medium having microorganisms capable of bioconverting syngas to oxygenated organic compounds, said fermenter being under fermentation conditions, to provide an aqueous medium containing said oxygenated organic compounds and an off gas; and
h. continuously or intermittently withdrawing at least a portion of the aqueous medium from the fermenter and recovering oxygenated organic compounds therefrom by distillation from the aqueous medium wherein steam from step (f) provides heat for the distillation.

In one preferred embodiment of this aspect of the processes of the invention, the hot exhaust stream, after providing indirect heat for gasifying the pyrolyzed coal, provides heat via indirect heat exchange for the thermal treatment of step (c).

In another broad aspect of this invention, processes are provided for the conversion of coal containing mercury compounds, hydrocarbon-containing compounds, sulfur-containing compounds and nitrogen-containing compounds to at least two types of chemicals, one of which is from hydrocarbon-containing compounds in the coal and another of which is an anabolic product from syngas fermentation, comprising:
a. drying particulate coal, preferably having a major dimension less than about 50 millimeters, preferably less than about 5 or 10 millimeters, at a temperature less than about 200° C. resulting in a water vapor overhead;
b. heating the dried coal to a temperature to between about 200° C. and 320° C. to remove mercury from the coal by volatilization and provide a heated coal;
c. thermally treating the heated coal in a pyrolysis zone at a temperature between about 430° C. to about 700° C. for a time sufficient to release vaporous organic compounds to provide a pyrolyzed coal having a reduced hydrogen content and a hot vapor stream containing hydrocarbon-containing compounds, said thermal treatment being sufficient to reduce the concentration of sulfur-containing and nitrogen-containing components by release from the coal being thermally treated, said thermal treatment including the passage of a sweep gas in the pyrolysis zone to minimize undue cracking of said hydrocarbon-containing compounds;
d. condensing hydrocarbon-containing compounds from said vapor stream to provide a first liquid product;
e. gasifying the pyrolyzed coal in the presence of steam under gasification conditions, said gasification conditions preferably comprising a temperature in the range of between about 800° C. and 1200° C. and a steam to carbon mole ratio of about 1.1:1 to 4:1, to provide an ash fraction and a syngas;
f. cooling by indirect heat exchange the syngas from step (e) and generate steam;
g. passing at least a portion of the cooled syngas to at least one fermenter containing an aqueous medium having microorganisms capable of bioconverting syngas to oxygenated organic compounds, said fermenter being under fermentation conditions, to provide an aqueous medium containing said oxygenated organic compounds and an off gas; and
h. continuously or intermittently withdrawing at least a portion of the aqueous medium from the fermenter and recovering the oxygenated organic compounds therefrom by distillation from the aqueous medium wherein steam from step (f) provides heat for the distillation.

A preferred embodiment of this aspect of the invention comprises using at least a portion of the off gas from step (g) as the sweep gas in the thermal treatment of step (c). In a more preferred aspect, the off gas is combusted before or during the thermal treatment to increase its temperature. In another preferred embodiment, oxygen is introduced during the thermal treatment of step (c) and is combusted to provide heat by direct heat exchange for the thermal treatment wherein the products of such combustion provide at least a portion of the sweep gas.

In another preferred embodiment, the temperature of the pyrolyzed coal passing from step (c) to step (e) is substantially maintained, i.e., is cooled by no more than about 50° C., or is increased, e.g., by indirect or direct heat exchange.

In some instances, the gasification is partially directly fired, and preferably air or oxygen-enriched air, say, containing at least about 50, preferably at least about 80, say 80 to 95, volume percent oxygen, is provided in the gasification as an oxidant. Preferably at least a portion of the first liquid product of step (d) is combusted with an oxidant in an indirect firing to provide a hot exhaust stream and at least a portion of heat energy in the hot exhaust stream of step (d) provides indirect heat for gasification. The indirect heat can be supplied by the hot exhaust stream by indirect heat exchange or by heating solids or fluids for introduction into the gasification step (e). Preferably, the indirect firing uses air or oxygen-enriched air, say, containing at least about 50, preferably at least about 80, volume percent oxygen, as an oxidant.

In another preferred embodiment, at least a portion of the condensed hydrocarbon-containing compounds of step (d) are subjected to hydrotreating. Hydrogen for the hydrotreating can be provided by at least one of the syngas and off gas from the fermentation of step (g).

In yet another embodiment, the water vapor overhead from step (a) is introduced into step (e) as at least a portion of the steam for the gasification.

### BRIEF DESCRIPTION OF THE DRAWING

FIG. 1 is a schematic depiction of a process in accordance with this invention.

FIG. 2 is a schematic depiction of another process in accordance with this invention in which essentially all of the volatilized hydrocarbonaceous component from a thermal treatment is used in a fired heater to provide indirect heat to the gasifier.

FIG. 3 is a schematic depiction of a linked thermal treatment kiln and fired heater for use in providing indirect heat to a gasifier.

### DETAILED DISCUSSION

All patents, published patent applications and articles referenced in this detailed description are hereby incorporated by reference in their entireties.

The use of the terms “a” and “an” is intended to include one or more of the element described. Lists of exemplary elements are intended to include combinations of one or more of the element described. The term “may” as used herein means that the use of the element is optional and is not intended to provide any implication regarding operability.

The term chemicals is intended to include organic compounds containing one or more carbon atoms which may be final products, intermediates or liquid fuel.

Oxygenated organic compounds are one or more organic compounds containing two to six carbon atoms selected from the group of aliphatic carboxylic acids and salts, alkanols and alkoxide salts, and aldehydes and can contain one or more oxygen-containing moieties such as diols, diacids and hydroxyethers and acids.

The release of a component from coal during thermal treatment contemplates both vaporization of the component and an occurrence of one or more chemical reactions to produce a component that is vaporous at the conditions of temperature, pressure and partial pressure of the thermal treatment.

References to organic acids herein shall be deemed to include corresponding salts and esters.

The processes of this invention are able to use various ranks of coal, and mixtures of coal and biomass can also be used. Although lower rank coals typically have higher water content, they also have higher hydrogen content and thus have a greater vaporizable organic component content. The preferred coals are bituminous and, most preferably, sub-bituminous, and have a volatile content of between about 10 and 50, more preferably between 20 and 45, mass percent with a hydrogen content between about 4 and 6 mass percent and an oxygen content between about 3 and 10 mass percent. Sub-bituminous coal is particularly useful where either chemicals from condensable organic compounds is sought or a substantial portion of the heat for the gasification is sought to be provided by indirect heating. Where mixtures of coal and biomass are used, often, the biomass constitutes up to about 40, say, between about 5 and 35, mass percent of the solid feed. The thermal treatment used in the processes of this invention can enable biomass to be used that contains phosphorus, nitrogen, sulfur and metal components that can be volatilized. Suitable solid biomass that can be used includes, but not limited to, animal and plant residues, municipal solid waste, and sludges from industrial and municipal wastewater treatments and from anaerobic digesters.

The processes of the invention can be used to produce chemicals via fermentation of syngas. The chemicals from the fermentation are oxygenated organic compounds and often comprise one or more of alkanols, carboxylic acids, carboxylic esters, ethers and carbonyls of from about 1 to 10, often 2 to about 6, carbon atoms. In some embodiments of the processes of this invention, chemicals are also obtained by vaporizing hydrocarbons contained in the coal and recovering hydrocarbon-containing fractions from the vapors.

The coal is sized for the thermal treatment and the gasification as is well known. Generally the coal is sized to be minus 50 millimeters, preferably minus about 20 millimeters, screen size (i.e., the coal particles pass through a screen of the designated size). In many instances, it is preferred to use coal having a particle size of less than about 1 millimeter in major dimension. The coal can optionally be subjected to a beneficiation treatment. Advantageously at least a portion of the free and bound water in the coal are recovered to provide water for use in the process. The water can be recovered by any convenient process. A preferred method for recovering water from the coal is by heating to a temperature below that where volatile mercury compounds are evolved, e.g., less than about 150° C. or 170° C., and preferably a temperature of at least about 120° C., by indirect heat exchange to provide steam. The pressure is often less than about 50 kPa gauge, say, in the range of about 50 to 150 kPa absolute. This steam is preferably introduced into the gasifier as at least a portion of the water requirement for the generation of syngas. The steam may contain some organic compounds volatilized with the water, and the vaporized organic compounds can be converted to syngas in the gasifier.

The processes of this invention reduce the volatile metals and sulfur-containing and nitrogen-containing components in the coal prior to the gasification and thus attenuate the syngas clean-up operations to provide a suitable syngas for use in the fermentation to produce oxygenated organic compounds. The processes of this invention preferably use a two-step pretreatment: first, a step (preheating) that removes volatile metals and metal compounds, especially mercury, and second, a step (thermal treatment) that volatilizes sulfur-containing and nitrogen-containing components and volatilizes at least a portion of the volatile hydrocarbons in the coal (i.e., provides a pyrolyzed coal with a reduced hydrogen content). Since volatile metal components are removed, the volatilized vapors in the second step often contain little, if any, non-volatile metal components. Thus the volatilized hydrocarbons from the thermal treatment can be recovered or combusted without undue environmental risk from metals.

The removal of volatile metal components in the first step is often done at a temperature of between about 200° C. and 320° C., and at these temperatures, water vapor is emitted from the coal. Some light hydrocarbons, if present in the coal, will also be passed to the vapor phase. Preferably the temperature in the first step is below a temperature that results in the vaporization of significant amounts of hydrocarbons. At these temperatures, most of the sulfur-containing and nitrogen-containing components remain in the coal. The heating may be by at least one of direct and indirect heat exchange. Usually the vaporous exhaust is contacted with sorbent to remove the volatilized metal component although other unit operations can be used.

The thermal treatment, which is at a temperature in the range of between about 430° C. to about 700° C., results in the vaporization of hydrocarbon-containing compounds in the coal as well as sulfur-containing and nitrogen-containing components. Accordingly, the treated, pyrolyzed coal has a reduced content of sulfur-containing and nitrogen-containing components and is therefore capable of providing an advantageous syngas for fermentation. The thermal treatment may be conducted in any suitable vessel, and vertical and horizontal kilns are preferred to minimize energy costs while providing controllable coal residence times in the vessel. The vessel may be indirectly heated, or partially directly heated where a portion of the coal or gas in the vapor phase is oxidized. The thermal treatment may be at a pressure between about 50 kPa and 500 kPa absolute, and is usually at a pressure slightly above ambient atmospheric pressure, e.g., between about 5 and 50 kPa gauge. The specific conditions used for the thermal treatment will be determined based upon the vessel, or kiln, used, and the amount of hydrocarbon sought to be removed from the coal. Often, the residence time of the coal in the pyrolysis zone is less than about 6 or 10 hours, say, between about 0.5 and 6 hours.

The vapors from the pyrolysis zone can be used as fuel for supplying heat to the gasification or as a source of additional chemicals. In this latter aspect, all or a portion of the vapors are subjected to hydrocarbon recovery unit operations. The condensable hydrocarbons in the vapors extend over a broad range of boiling points, e.g., from about 125° C. to 550° C. or more. In this embodiment, a sweep gas is preferably used in the thermal treatment to avoid undue cracking of volatilized components by reducing the residence time of the vapors at the elevated temperature. Preferably the average residence time of the vapors in the pyrolysis zone is less than about 10 or 20 minutes, and is often in the range of about 10 or 30 seconds to 5 minutes. As typical pyrolysis zones exhibit a temperature profile, the average residence time of the gases at the peak of the temperature profile is sometimes less than about 10 or 20 seconds. Any suitable sweep gas may be used provided that it is not adverse to the recovery of the hydrocarbons. Examples of sweep gases include steam, which can be condensed and separated from the hydrocarbons, and off gas from the fermentation which contains water vapor, carbon dioxide and unreacted hydrogen and carbon monoxide. If used as a sweep gas, the off gas can be combusted before or during its residence in the vessel for the thermal treatment, or can be passed through the vessel and be contained in the vapors removed from the vessel. The rate that the sweep gas introduced will affect the average residence time of the gases in the pyrolysis zone.

The hydrocarbon recovery operations comprise a condensation of one or more hydrocarbon-containing fractions to provide a liquid product. These fractions may be treated, e.g., by hydrotreating, to remove sulfur and nitrogen-containing components, although where used as a feed to a refinery, any sulfur and nitrogen removal may be done by unit operations in the refinery. See, for instance, Ebbe Skov, et al., “CCI Reference Plant Design for Conversion of LRC to Clean-Coal Fuel and Synthetic Crude Oil, presented at the 11^{th} EUEC Energy & Environmental Conference, Tucson, Ariz., Jan. 29, 2008, and Chen Jin-qing, et al., “CCI Upgrading Technology of Low Rank Coal in the USA and its Economic Evaluation”, Scientific Research, 2010, The Second China Energy Science Forum.

Hydrocarbon fractions not used in making a liquid product can be used as fuel for providing indirect heat for gasification. Thus, in this aspect of the processes of the invention, the value of the condensable hydrocarbon components can be maximized for a given market and location circumstance with the remaining hydrocarbons providing energy for the conversion of coal to syngas and then to an anabolic liquid product.

Alternatively substantially all of the vapors from the thermal treatment are combusted to provide heat for the gasification of the coal. In this embodiment, the temperature of the vapors is substantially maintained at temperatures which do not enable any appreciable condensation of hydrocarbon-containing compounds to avoid plugging of piping. Frequently, the exit temperature of the vapors from the pyrolysis zone is less than the peak temperature in the pyrolysis zone which enables some reduction of temperature to occur as the gases are being transferred to the combustion unit operation. Hence, the temperature of the vapors are preferably at or above the exit temperature of the vapors from the thermal treatment, e.g., the vapors are not cooled to a temperature less than about 100° C., preferably not less than about 50° C., of the exit temperature. Preferably the duration of time between the exit of the vapors from the thermal treatment to the point of combustion is rapid, for instance, less than about 10 seconds such that undue polymerization of any active radicals in the vapors does not occur.

Any suitable coal gasification process can be used provided that at least a portion of the heat for gasification is provided indirectly by combustion of hydrocarbon-containing components that are obtained from the thermal treatment vapors. The indirect heat transfer from the combustion can occur in various ways. For instance, the combustion can provide hot gas for indirect heat transfer to the vessel used for gasification. Alternatively, the hot gas from the combustion can be used to heat a solid heat transfer medium that is introduced into the vessel for the gasification. The latter provides advantages in that the design of the vessel for the gasification is less complex to obtain good distribution of heat within the vessel and the temperature, and in that rate of supply of the hot heat transfer media can be better controlled to reflect changes in feed rates of coal. In most instances, some direct firing is required to provide sufficient heat for the gasification, and the amount of direct firing will depend upon the hydrocarbon content of the coal fed to the process and the amount of hydrocarbon vaporized in the thermal treatment and used for the combustion of indirect firing. Preferably at least about 50, and more preferably, at least about 70, percent of the thermal energy for the gasification is obtained by indirect firing.

In the preferred aspects of the process of this invention, the pyrolyzed coal from the thermal treatment is introduced into the vessel for gasification without cooling, e.g., the temperature of the coal is not reduced by more than about 100° C., preferably no more than about 50° C., below its exit temperature from the thermal treatment. In some instances it may be desired to combust a portion of the coal prior to its introduction into the vessel for gasification to increase the temperature of the coal and reduce the amount of direct and indirect heat exchange required for the gasification. This combustion can be done in the thermal treatment or in a combustion zone between the thermal treatment and the gasification. The combustion gases from any such combustion zone can be used as a source of indirect heat for unit operations such as the thermal treatment, preheating or drying the coal. Accordingly, the presence of nitrogen in the oxidizing gas would not adversely affect the hydrogen and carbon monoxide concentrations in the syngas. The coal from the thermal treatment is reactive due to having increased surface activity and is often pyrophoric enabling combustion to readily occur. Additionally, a surface oxidation of the coal, particularly for sticky coals, retards or prevents undue agglomeration.

The conditions used for gasification can vary over a wide range and are not critical to the processes of this invention. Typically, the temperature of the gasification is within the range of between about 800° C. and 1200° C. The use of coal from which condensable volatiles have been removed often enables higher temperatures to be used for the coal gasification without undue generation of unsaturated hydrocarbons such as ethylene, acetylene, benzene, toluene, cumene, and higher aromatics. The pressure of the gasification can also vary over a wide range and is usually about 90 to 1000, say, about 110 to 500, kPa absolute. The average residence time of the coal in the gasifier is sufficient to convert at least about 90, preferably at least about 95, percent of the carbon in the coal fed to the gasifier to carbon oxides (carbon monoxide and carbon dioxide) especially where the gasifier is partially directly fired. The ratio of water to coal for the gasification can also fall within a broad range, and water is frequently in a molar excess, say, about 1.1:1 to about 4:1 moles of water per atom of carbon in the coal. The mole ratio of hydrogen to carbon monoxide in the syngas can vary over a wide range and is usually between about 0.5:1 to about 3:1. Frequently, the hydrogen and carbon monoxide constitute at least about 50, preferably at least about 70, volume percent of the syngas.

As stated above, at least a portion of the vapor from the thermal treatment is combusted to provide indirect heat for the gasification. Any suitable apparatus may be used for the combustion. In embodiments where the vapor is combusted without cooling, higher combustion temperatures can be achieved with a concomitant increase in the amount of thermal energy that can be transferred. In some instances, oxygen-enriched air, where available, may be used to increase the combustion temperature. A venturi-type burner where the oxidizing gas is used as the motive fluid, can be used to facilitate mass transfer of the vapor to the combustion apparatus. The combustion gases, after being used to provide heat for the gasification, contain sufficient thermal energy that they can be used for indirect heat exchange with one or more upstream unit operations, including the thermal treatment, pretreatment and drying. The use of multiple unit operations and their temperatures of operation facilitate a cascading of indirect heat exchange. Any sulfur oxides or nitrogen oxides can be removed, if desired, from the combustion gases after use as an indirect heat exchange fluid by any suitable means. If desired, carbon dioxide can be recovered and used as a feed to the syngas fermentation where the fermentation pathway requires both hydrogen and carbon dioxide for metabolically making the liquid product. In this case, additional hydrogen than that generated by the gasification of coal would be required to bioconvert any added carbon dioxide.

The syngas is cooled, treated to remove components that could be adverse to the fermentation, and used as substrate for the fermentation to make liquid product. The syngas cooling is done in part by indirect heat exchange in a steam boiler to provide steam for process purposes including, but not limited to, reboiler heating in a distillation operation to separate liquid product from water. Condensate from the syngas may be recovered for use in the process. Methods to remove undesired components from syngas are known in the art and the component to be removed and the extent of its removal will be dependent upon the nature of the microorganism used for the fermentation.

For the bioconversion of syngas to many oxygenated organic compounds, the ration of hydrogen to carbon oxides in the syngas from the coal gasification is too low to bioconvert all carbon oxides. Hence, in some preferred embodiments of the processes of this invention, the syngas from the gasification of coal is supplemented with hydrogen rich gas. The relative rate that the supplemental hydrogen is provided will depend upon the sought bioconversion of carbon oxides and thus can vary widely. For the bioconversion of syngas to ethanol, 2 moles of hydrogen per mole of carbon monoxide are required to avoid a net production of carbon dioxide, and 3 moles of hydrogen are stoichiometrically required to bioconvert carbon dioxide. The supplemental hydrogen can be provided by any convenient source. Steam reforming of methane, for instance, provides a higher ratio of hydrogen to carbon oxides than required, for instance, to bioconvert carbon monoxide to ethanol, and can be a net provider of hydrogen. Other sources of hydrogen include electrolysis of water and thermochemical cycles such as based on sulfur and iodine.

In embodiments of this invention where a hydrocarbon-containing liquid fraction from the vapor provided by the thermal treatment is hydrotreated, hydrogen may be supplied by the syngas or the off gas from the fermentation, especially where supplemental hydrogen is added to the syngas. If desired, the concentration of hydrogen can be increased using any suitable technique such as membrane separation.

The fermentation is conducted under suitable anaerobic fermentation conditions including the presence of suitable microorganisms. Numerous anaerobic processes are known for the conversion of syngas to liquid products. The fermentation conditions will depend upon the nature of the microorganism used for the conversion. The microorganism may be a wild-state microorganism or genetically modified, e.g., through recombinant procedures. The fermentation conditions are often mesophilic or thermophilic, and are anaerobic. See, for instance, United States published patent applications 2013/0109066; 2013/0137151; 2012/0309066; 2013/0005014; and 2013/0005021 and U.S. Pat. Nos. 8,354,257 and 8,309,348, all of which are hereby incorporated by reference in their entireties.

The fermentation provides an off gas containing unreacted substrate and inerts. In many instances, the syngas from the gasification of coal will contain methane, which is inert to the bioconversion. Substrate can be recovered and recycled or passed to a sequential fermentation. In some instances the off gas is used to provide hydrogen for hydrotreating or as a sweep gas for the thermal treatment or may be used for its energy value.

Recovery of the liquid product may be any suitable unit operation assembly, one unit operation of which is a distillation to separate liquid product from water.

The oxygenated organic compounds find utility as fuels and as chemical products and chemical intermediates for chemical products, fine chemicals and polymers. The oxygenated organic compounds can find application as liquid fuels as fuel replacements and additives such as ethanol and butanol. Ethanol may also be used as an intermediate to make butanol, see, for instance, United States Published Patent Application 2013/0116481. Butanol can also be used as a chemical intermediate to make chemicals, e.g., by dehydration and alkylation. Other applications of ethanol include, but are not limited to, dehydration to make ethylene and making butadiene.

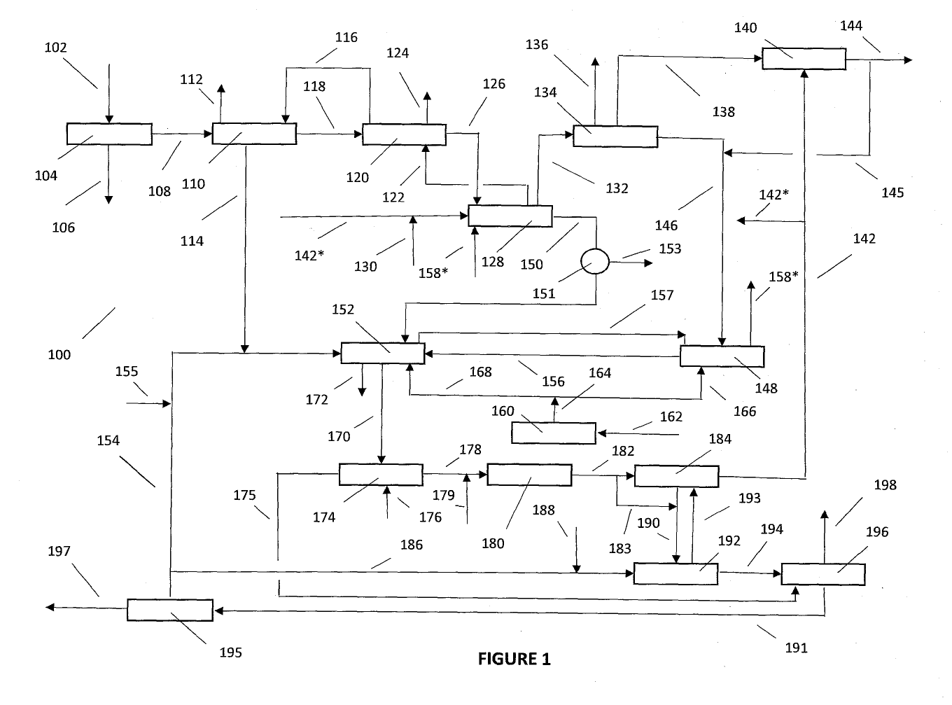

The processes of this invention will be described in further detail with reference to the drawings. Although FIGS. 1 to 3 facilitate the understanding of the broad aspects of the processes of this invention, they are not in limitation of the invention. The Figures omit minor equipment such as pumps, compressors, valves, instruments and other devices the placement of which and the operation thereof are well known to those practiced in chemical engineering. The Figures also omit ancillary unit operations.

FIG. 1 which schematically depicts apparatus 100. Coal is introduced into apparatus 100 via line 102 in to coal pretreatment operation 104. In coal pretreatment operation 104, coal is crushed to an appropriate size and washed. Typically, the crushing of the coal provides particles less than about 50 millimeters in major dimension, although other sizes can be used. Washing the coal can separate a portion of the clay, ash and inorganic sulfur associated with the coal. The waste from the washing operation is removed from coal pretreatment operation 104 via line 106. The washed and pulverized coal is then passed via line 108 to dryer 110. As shown, dryer 110 is indirectly heated using a heating fluid provided via line 116 which will be described in further detail later. Cooled heating fluid exits dryer 110 via line 112. The advantage of an indirect heated dryer is that the coal during drying provides a relatively concentrated moisture containing stream which can be used to supply at least a portion of the water for gasification of the coal as described below. This moisture-containing stream is withdrawn from dryer 110 via line 114. Frequently, the temperature of the drying is sufficient to provide a coal containing less than about 5, preferably less than about 3, mass percent moisture. The temperature of the drying is often at least about 120° C., and is often below about 200° C. It is possible that volatile hydrocarbons such as methane may be contained in the moisture containing stream. Since the moisture-containing stream is introduced into a gasifier, as described later, these volatile hydrocarbons will be gasified. The drier will also contain adventitious air which will pass with the steam to the gasifier and provide some oxygen for reaction with the coal for direct heat generation.

The dried coal is then passed to preheater 120 via line 118. As shown, preheater 120 is indirectly heated using a heating fluid provided via line 122. The cooled heating fluid exits via line 116 and is passed to dryer 110. Mercury-containing compounds are volatilized and removed with the gases exiting in line 124. The mercury can be recovered by any suitable unit operation. In preheater 120 the coal is raised to a temperature between about 200° C. and 320° C. With the use of coals having a significant swelling index (e.g., bituminous coals having a swelling index of 4 or more), it is sometimes desired to add an oxygen-containing gas such that the surface of the coal particles are oxidized to defeat swelling or stickiness.

The preheated coal is then passed via line 126 to thermal treatment kiln 128. Although one thermal treatment kiln 128 is depicted in FIG. 1, typical commercial-scale facilities will contain a plurality of kilns. Thermal treatment kiln 128 serves to increase the temperature of the coal to between about 430° C. to about 700° C., preferably 500° C. to 650° C. At these temperatures, various organic compounds are released as vapors including condensable C5+hydrocarbons. Typically, sulfur compounds including hydrogen sulfide, sulfur-containing organic compounds and carbonyl sulfide pass to the vapor phase as well as ammonia and other nitrogen-containing compounds.

As shown, the thermal treatment is effected by direct and indirect heat exchange. A sweep gas is introduced into thermal treatment kiln 128 via line 142*. (The asterisk indicates that the lines having the same numerical designation are connected but not shown in the drawing for purposes of clarity.) This is a hydrogen and carbon monoxide-containing off gas from the fermentation and is combined with air supplied via line 130 for combustion to provide direct heat transfer to thermal treatment kiln 128. A combustion gas is supplied via line 158* for indirect heat exchange. Thermal treatment kiln 128 may be a vertical kiln or a horizontal kiln, especially a horizontal rotating kiln.

A vaporous stream is removed from thermal treatment kiln 128 via line 132 and is passed to condensation and separation unit operations 134. A lights-containing stream is withdrawn from condensation and separation unit operations 134 via line 136. This lights-containing stream can be passed to a thermal oxidizer (not shown) to combust any hydrocarbon containing material, hydrogen and carbon monoxide present. The condensed fraction often has a wide range of boiling points, e.g., from about 125° C. to 550° C. or more. As shown, a condensed fraction from the vapor stream which contains hydrocarbons, nitrogen-containing compounds and sulfur-containing compounds is passed via line 138 to hydrotreater 140. Hydrogen for the hydrotreating is provided by line 142 as will be discussed later, and a hydrotreated hydrocarbon product is withdrawn via line 144. If desired, more than one hydrocarbon fraction can be produced, and an especially valuable fraction is an ultralow sulfur diesel fraction. Higher and lower boiling fractions may be used in further petroleum refining operations such as reforming and cracking, or may be used as a source of energy for gasification. As shown, line 145 is provided to optionally direct all or a portion of the hydrocarbon product for use as a fuel for the indirect firing of the gasifiers. However, preferably essentially all of the hydrocarbon product is withdrawn via line 144 for marketing.

Returning to thermal treatment kiln 128, considerable latitude exists in respect of the portion and boiling range of the vaporizable components are removed from the coal since the value of any residual vaporizable components will be captured during the gasification. This is in contrast to situations where the coal is to be used, e.g., for combustion to produce heat or power, since a residual amount of hydrocarbon is desired to facilitate light off of the coal. Accordingly, the operator has flexibility to optimize the integrated process for sought objectives. For instance, where maximizing the production of hydrotreated hydrocarbon product, the operator can choose to operate the thermal treatment kiln to increase the amount of condensable hydrocarbons that are vaporized. On the other hand, where the bioconversion of syngas is to be maximized, the operator may choose to operate the thermal treatment kiln such that a lesser portion of condensable hydrocarbons are vaporized, but in amounts sufficient to provide a significant portion of the heat for the indirect firing of the gasifier when combusted. In either event, an important function of the thermal treatment is to enhance the reactivity of the coal in the subsequent gasification unit operation. It is believed that the thermal treatment serves to increase the surface area of the coal, and in some instances, the pyrolyzed coal may become self-catalytic.

Where an indirect, or partially indirect heating of the gasifier is used, the operator can recover vaporizable components for purposes of combustion to provide indirect heat. Thus, the operator can, by varying the conditions of the thermal treatment, secure a desired volume of condensable hydrocarbons to supply to a fired heater. As shown, line 146 is adapted to direct condensed hydrocarbons from condensation and separation unit operation 134 to fired heater 148. In the event that the condensed hydrocarbons contain undesirable amounts of sulfur, it is preferred that the condensable hydrocarbons be subjected to hydrotreating or the combustion gases be subjected to sulfur dioxide removal treatment. Thus the hydrocarbons passing to fired heater 148 may be obtained from hydrotreater 140 via line 145.

Pyrolyzed coal, from which volatile components are removed, exits thermal treatment kiln 128 via line 150 and is directed to gasifier 152. It is generally desired to use more than one gasifier 152 such that a continuing supply of syngas can be maintained to the fermenter in the event that one gasifier fails or is brought down for repair or inspection. Additionally, the use of more than one gasifier 152 facilitates ramp-up during startup. Typically, thermal treatment kiln 128 and gasifier 152 are close coupled to minimize cooling of the coal. In some instances, it may be desired to provide an ash-removal unit operation prior to introducing the coal into the gasifier. Thus, as shown, ash-removal unit operation 151 is provided in line 150 to remove a portion of the ash via line 153. Ash-removal unit operation 151 may be any suitable unit operation, including, but not limited to, high temperature magnetic separation.

In gasifier 152, coal and water, which is provided via line 154, are reacted to provide a syngas containing carbon monoxide and hydrogen. The syngas will also contain other components such as carbon dioxide and potentially hydrocarbons and sulfur and nitrogen containing compounds if sulfur and nitrogen are present in the coal. Gasifier 152 may be any suitable gasifier for generating syngas from coal, and can be a directly-fired gasifier, an indirectly-fired gasifier or a partially, indirectly-fired gasifier. Where gasifier 152 is directly fired or is partially directly fired, it is usually desired to use oxygen or oxygen-enriched air for the combustion such that the syngas does not become unduly diluted with nitrogen. As shown, gasifier 152 is partially directly fired, and oxygen-enriched air is provided by membrane air separation unit 160 via line 164 and header 168.

Indirect heat is provided by fired heater 148. Fired heater 148 may be of any suitable type. For instance, and not in limitation, fired heater 148 can be in indirect heat exchange with the interior of gasifier 152. Alternatively, fired heater 148 can heat a gas that can be used in direct or indirect heat exchange with the interior of gasifier 152, and this gas can be steam or other suitable gas for introduction into gasifier 152. Line 156 conducts the heated gas to gasifier 152, and where the gas is used in indirect heat exchange, line 157 returns the gas to fired heater 148. And yet another alternative, fired heater 148 heats solids that are then transported via line 156 to gasifier 152, and the solids are returned to fired heater 148 via line 157. The solids that are used for transporting heat (thus providing indirect heat) to gasifier 152 are preferably composed of heat resistant materials such as quartz and alumina and are of a size that facilitates separation of the coal being gasified and its ash from the solids used for heat transfer.

Although optional, fired heater 148 is shown as using oxygen-enriched air from the air separation unit 160. The oxygen-enriched air is supplied from line 164 via header 166. An advantage of using oxygen-enriched air for the combustion is that higher temperatures can be achieved. Hot combustion gases exit fired heater 148 via line 158*. These hot combustion gases are passed to thermal treatment kiln 128 via line 158*.

Ash is withdrawn from gasifier 152 via line 172. The ash can be solid or molten depending upon the type of coal used and the design and operating conditions of the gasifier. In any event, it is preferred that the coal and the gasification conditions do not produce sticky ash particles that can cause build-ups in the gasifier.

Syngas is exhausted via line 170 from gasifier 152 and passes to steam boiler 174. Water enters steam boiler 174 through line 176. Steam is withdrawn via line 175 use in heat exchange in the process of this invention. Any suitable exchanger or assembly of heat exchangers may be used for steam boiler 174 as is well known in the art. Preferably the syngas is cooled to a temperature below about 150° C., more preferably below about 125° C. In one option, the cooling is to temperature sufficiently low that water is condensed, and the condensed water may be recycled to line 154 for reintroduction into gasifier 152. The condensed water will contain soluble components in the syngas such as carbon dioxide, and may contain some hydrocarbons as well as sulfur-containing compounds and nitrogen-containing compounds.

The cooled syngas is passed from heat exchanger 174 to purification unit operations 180 via line 178. As shown, syngas from a steam methane reformer is introduced via line 179 and is combined with the cooled syngas. Although shown as being combined prior to the syngas purification, in many instances the syngas from steam methane reformers need not be subjected to purification operations and can be combined with the syngas from the coal gasification after the purification operations.

Purification operations 180 may be any suitable purification unit operations required to provide syngas suitable for introduction into a fermentation broth. Thus, the purification operations can include one or more unit operations to remove nitrogen oxides, hydrogen cyanide, and other components deleterious to the microorganisms used in the fermentation.

The purified syngas exits purification operations 180 via line 182. The syngas may be directly introduced into fermenter 192 via line 182 and lines 183 and 190. Fermenter 192 is typically a plurality of bioreactors. Alternatively, all or a portion of the syngas in line 182 may be directed to syngas separator 184 to provide a hydrogen-rich stream that can be passed via line 142 to hydrotreater 140. As shown, a portion of the gas in line 142 is withdrawn via line 142* which connects with line 142* that provides sweep gas to thermal treatment kiln 128. Syngas separator 184 may be of any convenient design as well known in the art including selective sorption and membrane enrichment unit operations. A stream having a lower mole ratio of hydrogen to carbon monoxide is withdrawn from syngas separator 184 via line 190 and is passed to fermenter 192. Although a portion of the syngas can be subjected to water gas shift to provide a higher hydrogen-content gas for the hydrotreating, the water gas shift results in carbon monoxide being converted to carbon dioxide. Since the fermentation bioconverts carbon monoxide, any unit operation reducing the availability of carbon monoxide for fermentation is less preferred. In more preferred operations, essentially all of the hydrogen is passed to fermenter 192 to maintain higher hydrogen partial pressure and thus stronger driving forces to dissolve hydrogen in the aqueous fermentation broth.

Also supplied to fermenter 192 is water and nutrients including micronutrients for the microorganisms. As shown, water is provided by line 186, and nutrients and makeup water is provided to line 186 via line 188.

Fermenter 192 is maintained under fermentation conditions including the presence of microorganisms suitable for converting syngas to oxygenated organic compound. For purposes of discussion, the liquid product is either ethanol or butanol (isobutanol and/or n-butanol) which have utility as chemicals.

Fermenter 192 may be of any suitable design including, but not limited to, bubble column, stirred reactor, jet loop reactor, membrane reactor and the like. The microorganisms may be in free suspension or located on a solid surface or in a solid. The supply of syngas to fermenter 192 is sufficient not only to maintain the population of microorganisms but also to produce the oxygenated organic compounds. An off gas is withdrawn from fermenter 192 via line 193. This off gas contains unreacted carbon monoxide, hydrogen and carbon dioxide and inert gases, such as methane and nitrogen, as well as water vapor. Due to the greater solubility of carbon monoxide in water as compared to the solubility of hydrogen, the off gases generally have a higher hydrogen to carbon monoxide mole ratio than the syngas entering fermenter 192. As shown, the off gases are directed by line 193 to syngas separator 184. Hence, all, or a portion of the hydrogen sought for the hydrotreating can be derived from the off gases thereby providing an operating efficiency of the integrated process. Diluents such as methane and nitrogen are tolerable in the hydrogen used for hydrotreating. At least a portion of the hydrogen and carbon monoxide not exiting the syngas separator via line 142 can be recycled to fermenter 192 via line 190.

Aqueous fermentation broth is withdrawn from fermenter 192 and is passed via line 194 to product recovery operations 196 for recovery of oxygenated organic compound from the broth. The liquid product is withdrawn via line 198, and a water-containing stream is withdrawn via line 191, from product recovery operations 196. Product recovery operations 196 may be any suitable unit operation or assembly of unit operations to effect separation of the liquid product such as, but not limited to, distillation, crystallization, phase separation, and selective sorption. As shown, steam from steam boiler 174 is passed via line 175 to product recovery operations 196 to provide heat for a distillation column to separate liquid product from water.

The water-containing stream in line 191 can contain solids, especially where fermenter 192 uses a free suspension of cells. As shown, the water containing stream is directed to waste treatment operations 195 from which a more purified water stream is obtained and withdrawn via line 154 and a waste stream is discharged via line 197. Waste treatment operations 195 can comprise any suitable unit operation or assembly of operations including, but not limited to, filtration, ultrafiltration, membrane separation, distillation, ion exchange, and anaerobic and/or anaerobic digestion. The more purified water stream in line 154 can be used in the process, e.g., by recycle to fermenter 192 via line 186, and to gasifier 152. As shown, supplemental water is provided via line 155 to the water in line 154 being passed to gasifier 152. The water passed to fermenter 192 should have an absence of free oxygen.

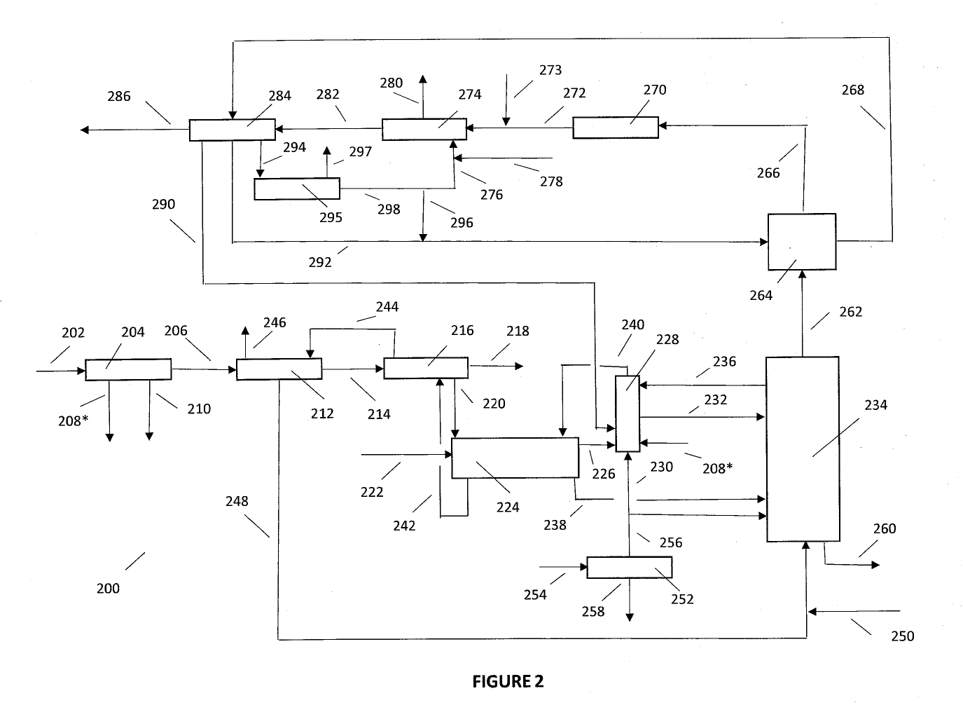

Apparatus 200 is depicted in FIG. 2 and is adapted to provide a single liquid product, ethanol, from coal. Coal is supplied via line 202 to coal pretreatment operation 204. In coal pretreatment operation 204, coal is crushed to an appropriate size and washed. The washed and pulverized coal is then passed via line 206 to dryer 212. Fine coal particles, often less than about 100 microns in major dimension are generated during the crushing of the coal, and they are recovered by filtration and are passed from coal pretreatment operation 204 via line 208* for use as supplemental fuel as will be described later. Washing the coal can separate a portion of the clay, ash and inorganic sulfur associated with the coal. The solid waste from the washing operation is filtered and removed from coal pretreatment operation 204 via line 210. The waste washing water can be recycled.

The coal is passed to dryer 212 which is operated at a temperature sufficiently low that volatile mercury components are not evolved. A low temperature steam overhead is produced which is transported via line 248 for use in the gasifier as described below. The coal still contains residual moisture and is passed via line 214 to preheater 216 which serves to provide a steam containing vapor stream which exits via line 218. Carried with the steam will be volatile metal components such as mercury. Mercury can be recovered from the stream in line 218 by any suitable means including selective sorption. The stream will also contain some vaporized hydrocarbonaceous materials. Depending upon the contaminants in the coal, and the temperature used for the preheating, the water vapor in line 218, after removal of mercury, maybe suitable for introduction into the gasifier as a source of water. However, if the temperature is sufficiently high that undue amounts of sulfur components are volatilized, the stream is preferably directed to plant wastewater treatment.

The coal is then transported from preheater 216 via line 220 to thermal treatment kiln 224. The temperature of the coal is increased to about 430° C. to about 700° C. such that a substantial amount of sulfur and nitrogen-containing components are volatilized along with hydrocarbonaceous materials. A sweep gas is provided to thermal treatment kiln 224 via line 222 to reduce the residence time of the vapors being evolved from the coal in thermal treatment kiln 224. The use of a sweep gas is not essential to the process depicted in FIG. 2 since the recovery of hydrocarbons is not being undertaken. Nevertheless, any cracking that occurs of the vaporous components will be endothermic. One suitable sweep gas is the off gas from the fermentation which contains hydrogen, carbon dioxide and some carbon monoxide as well as inerts. The off gas can be combusted prior to entry into thermal treatment kiln or in thermal treatment kiln to provide heat for the thermal treatment, or the off gas can be passed through thermal treatment kiln 224 and combusted in fired heater 228.

The vapor from thermal treatment kiln 224 exits via line 226 and is passed to fired heater 228. Oxidant is provided to fired heater 228 via line 230. The oxidant may be air or oxygen-enriched air. Advantageously, the vapor from thermal treatment kiln 224 is maintained substantially at temperature. This provides two advantages. First, the condensation of any vapor is substantially avoided thereby reducing maintenance, and second, a higher flame temperature can be obtained in fired heater 228 which can impart more heat energy for indirect heating of the gasifier. Fired heater 228 heats up solid heat transfer media which are passed to gasifier 234 via line 232, and cooled heat transfer media are returned to the fired heater from gasifier 234 via line 236. The recycle rate of the heat transfer media is such that indirect heating of the gasifier occurs, and the gasifier is maintained at a desired temperature range to effect the generation of syngas without the production of undue amounts of side products such as aromatics. The recycle rate will also determine the maximum temperature of the heat transfer media with slower rates resulting in the media approaching the maximum combustion temperature in fired heater 228.

The amount of heat that can be provided by fired heater 228 will be related to the amount of fuel available for combustion. As discussed above, thermal treatment kiln 224 can be operated over a range of temperatures and coal residence time for a given coal containing a given amount of hydrocarbonaceous volatiles. The amount of hydrocarbonaceous volatiles removed from the coal can be varied by changing temperature and residence times, and thus affect the amount of fuel available for the fired heater. All or a portion of the fines generated in coal pretreatment operation 204 can be passed via line 208*to fired heater 228 as supplemental fuel. Additionally, undesired vaporous byproducts from the fermentation may be passed via line 290 to fired heater 228 as additional fuel. Thus, depending upon the volatiles content of the coal feedstock, the indirect heating of the gasifier provided by fired heater 228 can provide at least a major portion if not all the required for the gasification. Thus, direct firing of gasifier 234 itself is a minor portion of the source of heat for the gasification, and consequently, the use of oxygen-enriched air can be used instead of pure oxygen without undue reduction in partial pressures of hydrogen and carbon monoxide in the syngas.

Hot combustion gases from fired heater 228 are used in heat exchange. First, the combustion gases are passed via line 240 to thermal treatment kiln 224 for indirect heat exchange. The combustion gases then exit thermal treatment kiln 224 via line 242 and are passed to preheater 216 for indirect heat exchange. The combustion gases then exit preheater 216 via line 244 and are used for indirect heat exchange in dryer 212. The cooled combustion gases are finally exhausted via line 246. Preferably the temperature of the finally exhausted combustion gases is maintained above that which results in any significant condensation of water.

Coal is passed via line 238 from thermal treatment kiln 224 to gasifier 234. The thermal treatment provides a coal with high surface activity and reactivity and thus facilitates gasification. Water, which may be in the form of steam, is provided to gasifier 234 via line 248 to which additional water is added via line 250. Air purification unit 252 is provided to supply oxygen-enriched air 256 to gasifier 234. Air purification unit 252 can comprise a membrane separation or pressure swing adsorption separation and often the oxygen-enriched air contains between about 50 and about 90 volume percent oxygen. As shown, air purification unit 252 also supplies oxygen-enriched air to fired heater 228. The use of oxygen-enriched air increases the combustion temperature and thus the amount of heat available for the indirect firing of gasifier 234. Alternatively, air is often a suitable oxidant where ample fuel is available for combustion in fired heater 228. Air is provided to air purification unit 252 via line 254, and nitrogen-enriched gases are exhausted via line 258.

Gasifier 234 operates at a temperature sufficient to provide syngas, and preferably at least about 90, and more preferably at least about 95, percent of the carbon in the coal passing to gasifier 234 is consumed. Ash is withdrawn via line 260 from gasifier 234, and a syngas product exits gasifier 234 via line 262. The syngas is hot and is passed to syngas reboiler 264. Cooled syngas exits reboiler 264 via line 266. Steam exits reboiler 264 via line 268 and is used as process steam including for distillation column reboiler in the product recovery operations as discussed below.

Line 266 directs the cooled syngas to purification unit operations 270 for the purpose of removing adverse components from the syngas. The purified syngas is then passed via line 272 to fermentation assembly 274. As shown, syngas from a steam methane reformer is provided via line 273 and admixed with the gasification syngas in line 272. In fermentation assembly 274 a fermentation broth contains microorganisms for the bioconversion of syngas to ethanol. Fermentation assembly 274 is maintained under fermentation conditions and provides an ethanol-containing aqueous stream which is withdrawn via line 282 and an off gas which contains unreacted syngas and carbon dioxide, and also contains inerts such as nitrogen and methane, is removed via line number 280. The off gas may be recycled or at least in part used as a sweep gas provided via line 222.

The ethanol-containing aqueous stream in line 282 is passed to ethanol recovery unit operations 284 which may comprise solid separations, distillation and other purification operations such as ultrafiltration and membrane separation. In one embodiment, ethanol recovery unit operations 284 comprises at least one distillation column for the separation of water from ethanol and steam from line 268 is used to heat the reboiler. An ethanol product is provided via line 286. The spent steam (condensate) is passed via line 292 to steam reboiler 264. Undesirable components produced during the fermentation are withdrawn via line number 290. An aqueous stream, which may contain solids from the microorganisms, is passed from ethanol recovery unit operations 284 via line 294 to water treatment facility 295.

Water treatment facility 295 may be any suitable water treatment facility, and often includes an anaerobic digestion. Waste materials from water treatment facility 295 exits via line 297 and purified water is recovered and is passed via line 298 to the process. This water may be returned to fermentation assembly 274 via line 276 or passed via line 296 to line 292 as makeup water for steam generation. Makeup water and nutrients for the fermentation are provided via line 278.

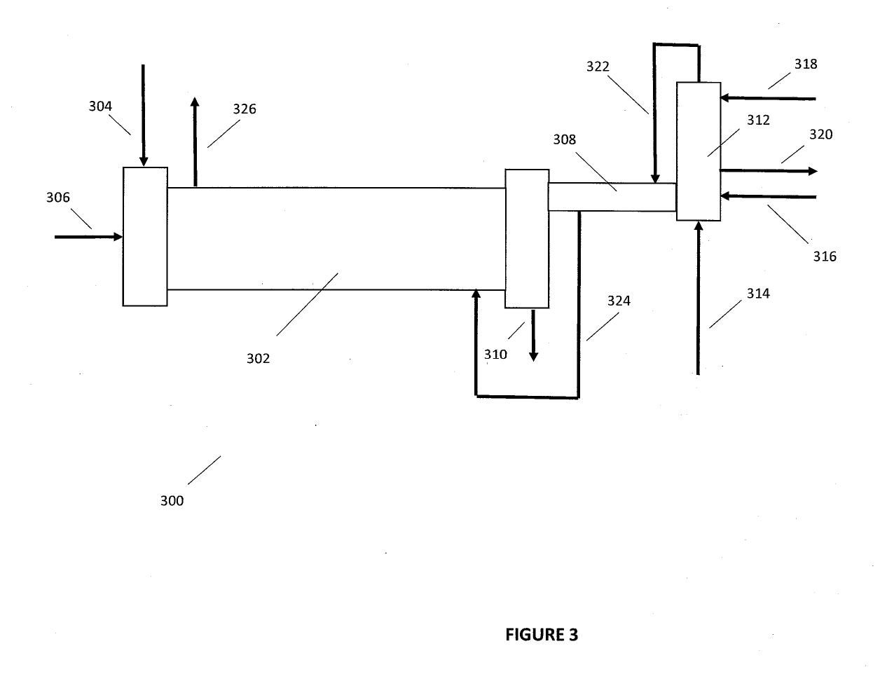

FIG. 3 is a schematic representation of a thermal treatment kiln and fired heater assembly which can be used in the processes of this invention, including, but not limited to those depicted in FIG. 1 and FIG. 2. The apparatus generally indicated as 300 comprises thermal treatment kiln 302 which is a jacketed, rotary kiln. Coal enters thermal treatment kiln 302 via line 304. A sweep gas is provided by line 306.

In thermal treatment kiln 302 a portion of the coal feed is vaporized and the vapor is withdrawn via jacketed line 308. The vapor is passed as feed to fired heater 312 in which it is combusted with an oxidant such as air or oxygen-enriched air from line 314. Additional combustible material may be provided to fired heater 312 via line 316. Fired heater 312 serves to increase the temperature of heat transfer media for use in a gasifier. The heat transfer media are provided via line 318 to fired heater 312 and are withdrawn via line 320 for return to the gasifier.

The combustion gases exit fired heater 312 via line 322 and are passed to jacketed line 308 to maintain the vapors from thermal treatment kiln 302 at or above the exit temperature of the vapors. The combustion gases are then passed via line 324 to the jacket of thermal treatment kiln 302 for the purpose of heating the coal to the sought temperatures. The combustion gases are then withdrawn via line 326.

It is claimed:

1. A process for the conversion of coal containing mercury compounds, hydrocarbon-containing compounds, sulfur-containing compounds and nitrogen-containing compounds to chemicals comprising:
a. drying particulate coal at a temperature less than about 200° C. resulting in a water vapor overhead;
b. heating the dried coal to a temperature to between about 200° C. and 320° C. to remove mercury from the coal by volatilization and provide a heated coal;
c. thermally treating the heated coal at a temperature between about 430° C. to about 700° C. for a time sufficient to release vaporous organic compounds to provide a pyrolyzed coal having a reduced hydrogen content and a vapor stream containing released organic compounds, said thermal treatment being sufficient to reduce the concentration of sulfur-containing and nitrogen-containing components in the coal being thermally treated;
d. combusting at least a portion of the vapor stream of step (c) to provide a hot exhaust stream wherein said at least a portion of the vapor stream is maintained substantially at or above the temperature of the thermal treating until combusted;
e. gasifying the pyrolyzed coal in the presence of steam under gasification conditions to provide an ash fraction and a syngas, wherein at least a portion of heat energy in the hot exhaust stream of step (d) provides indirect heat for gasification;
f. cooling by indirect heat exchange the syngas from step (e) and generate steam;
g. passing at least a portion of the cooled syngas to at least one fermenter containing an aqueous medium having microorganisms capable of bioconverting syngas to oxygenated organic compounds, said fermenter being under fermentation conditions, to provide an aqueous medium containing said oxygenated organic compounds and an off gas; and
h. continuously or intermittently withdrawing at least a portion of the aqueous medium from the fermenter and recovering oxygenated organic compounds therefrom by distillation from the aqueous medium wherein steam from step (f) provides heat for the distillation.

2. The process of claim 1 wherein the gasification is partially directly fired.

3. The process of claim 2 wherein the oxidant for the direct firing comprises air or oxygen-enriched air.

4. The process of claim 1 wherein the water vapor overhead from step (a) is introduced into step (e) as at least a portion of the steam for the gasification.

5. The process of claim 1 wherein the hot exhaust stream, after providing indirect heat for gasifying the coal, provides heat via indirect heat exchange for the thermal treatment of step (c).

6. The process of claim 1 wherein the oxygenated organic compounds comprise at least one of ethanol and butanol.

7. The process of claim 6 wherein the oxygenated organic compounds comprise ethanol and ethanol is converted to at least one of ethylene and butadiene.

8. The process of claim 1 wherein the syngas from the gasification of pyrolyzed coal is admixed with hydrogen.

9. The process of claim 8 wherein the hydrogen is contained in a hydrogen-rich syngas.

10. The process of claim 9 wherein the hydrogen-rich syngas is produced by the steam reforming of methane.

11. A process for the conversion of coal to at least two liquid products comprising:
a. drying particulate coal at a temperature less than about 200° C. resulting in a water vapor overhead;
b. heating the dried coal to a temperature to between about 200° C. and 320° C. to remove mercury from the coal by volatilization and provide a heated;
c. thermally treating the heated coal in a pyrolysis zone at a temperature between about 430° C. to about 700° C. for a time sufficient to release vaporous organic compounds to provide a pyrolyzed coal having a reduced hydrogen content and a hot vapor stream containing hydrocarbon-containing compounds, said thermal treatment being sufficient to reduce the concentration of sulfur-containing and nitrogen-containing components by release from the coal being thermally treated, said thermal treatment including the passage of a sweep gas in the pyrolysis zone to remove said hydrocarbon-containing compounds prior to the occurrence of undue cracking;
d. condensing organic compounds from said vapor stream to provide a first liquid product;
e. gasifying the pyrolyzed coal in the presence of steam under gasification conditions to provide an ash fraction and a syngas;
f. cooling by indirect heat exchange the syngas from step (e) and generate steam;
g. passing at least a portion of the cooled syngas to at least one fermenter containing an aqueous medium having microorganisms capable of bioconverting syngas to oxygenated organic compounds, said fermenter being under fermentation conditions, to provide an aqueous medium containing said oxygenated organic compounds and an off gas; and
h. continuously or intermittently withdrawing at least a portion of the aqueous medium from the fermenter and recovering oxygenated organic compounds therefrom by distillation from the aqueous medium wherein steam from step (f) provides heat for the distillation.

12. The process of claim 11 wherein oxygen is introduced during the thermal treatment of step (c) and is combusted to provide heat for the thermal treatment wherein the products of such combustion provide at least a portion of the sweep gas.

13. The process of claim 11 wherein the temperature of the coal passing from step (c) to step (e) is substantially maintained.

14. The process of claim 11 wherein at least a portion of the first chemicals of step (d) is combusted and serves as an indirect firing for a source of heat for the gasification of step (e).

15. The process of claim 14 wherein the indirect firing provides heated solids or fluids for introduction into the gasification step (e).

16. The process of claim 11 wherein at least a portion of the condensed organic compounds of step (d) are subjected to hydrotreating.

17. The process of claim 11 wherein the syngas from the gasification of pyrolyzed coal is admixed with hydrogen.

18. The process of claim 17 wherein the hydrogen is contained in a hydrogen-rich syngas.

19. The process of claim 18 wherein the hydrogen-rich syngas is produced by the steam reforming of methane.

20. The process of claim 19 wherein at least a portion of the condensed organic compounds of step (d) are subjected to hydrotreating and at least a portion of hydrogen for the hydrotreating is obtained from the off gas from step (g).
